(19) United States
(12) Patent Application Publication
(10) Pub. No.: US 2026/0007651 A1
(43) Pub. Date: Jan. 8, 2026

(54) Effect of YEL002 on Conditions and Diseases

(71) Applicant: RadMit, Inc., Los Angeles, CA (US)

(72) Inventor: Robert H Schiestl, Los Angeles, CA (US)

(21) Appl. No.: 19/281,708

(22) Filed: Jul. 27, 2025

Related U.S. Application Data
(63) Continuation of application No. 18/045,152, filed on Oct. 8, 2022, now abandoned.

(51) Int. Cl. A61K 31/4709

(52) U.S. Cl. CPC A61K 31/4709

(57) ABSTRACT

A preparation comprising the YEL002 compound as an active ingredient for the prevention or treatment of one or more diseases, and methods of using such preparations. The preparations may be a sublingual pill, an injection, dissolved in liquid, dissolved in oil, or any other preparation. The conditions or diseases indicated may be poisoning, radiation poisoning, chemotherapy poisoning, poor vision, peripheral artery disease, smoker's leg, diabetic feet, periodontal disease, varicose or spider veins, blood clots, COVID-19, long COVID, liver disease, symptoms caused by chemotherapy or radiation therapy, hair loss, cancer, inflammation-caused or inflammation-related diseases, autoimmune diseases, infections, and headaches including migraines.

### CROSS-REFERENCE TO RELATED APPLICATIONS

This application is a continuation of U.S. application Ser. No. 18/045,152 filed Oct. 8, 2022, and the entire content of prior-filed applications is incorporated by reference herein.

### BACKGROUND OF THE INVENTION

Below are summaries of conditions that afflict the human population and could be prevented, mitigated or cured by the compound YEL002.

Eye sight: There are about 2.2 billion people worldwide with poor vision which is almost 30% of all people. This is an underestimate since in many under-developed countries slightly reduced vision is not treated.

Smoker's legs and diabetic feet: Smoker's leg refers to symptoms in the leg resulting from a condition that doctors call peripheral artery disease (PAD). PAD causes narrowing of the blood vessels, which reduces blood flow to the arteries in the leg. It can also affect blood flow to the: arms, legs and feet, stomach and brain. About 6.5 million people which is about 2% have PAD in the United States. Diabetics have similar problems lower in the feet. The final therapy after many years of disease and pain involves amputation of legs and feet since necrosis develops that otherwise may affect the entire body.

Periodontics: Periodontics or receding gum line affects 20-50% of the entire population. Type 2 diabetic individuals with severe form of periodontal disease have 3.2 times greater mortality risk compared with individuals with no or mild periodontitis.

Varicose and spider veins: Approximately 23% of US adults have varicose veins. If spider telangiectasias and reticular veins are also considered, the prevalence increases to 80% of men and 85% of women. Generally, more common in women and older adults, varicose veins affect 22 million women and 11 million men between the ages of 40 to 80 years.

Blood clots: 900,000 people per year in the US have deep blood thrombosis and up to 25% of them die suddenly resulting in 100,000 death annually in the US only per year.

COVID19:600 million cases, 6.5 million death worldwide as of the end of August 2022.

Long Covid: About 25% of all people have long COVID which is about 1.9 billion people worldwide.

Liver disease: About 1.5 billion people worldwide have clinical liver disease.

Chemo and radiation therapy for cancer: In the US about 1.1 million people receive chemo or radiation therapy for cancer each year.

Hair loss: More than 80 percent of men and nearly half of women experience significant hair loss during their lifetime.

Cancer: Cancer is a disease that develops when cells in your body divide at a faster rate than normal cells. These abnormal cells grow into a lump—or tumor. About 25% of all people have cancer in their lifetime.

Inflammation-caused diseases: Inflammation is the cause of most diseases and about 51% of all people die of inflammation caused diseases.

Autoimmune disease: About 6% of all people have autoimmune diseases which is about 45 Million people worldwide.

Infections: Infectious diseases are the second leading cause of death worldwide, after heart disease, and are responsible for more deaths annually than cancer.

Headache: 75% of all people have headache in one year and 30% have migraines which can also be due to inflammation.

### BRIEF SUMMARY OF THE INVENTION

Disclosed are preparations comprising the YEL002 compound as an active ingredient for the prevention or treatment of one or more diseases. The preparations may be a sublingual pill, an injection, dissolved in liquid, or dissolved in oil. The conditions or diseases indicated may be poisoning, radiation poisoning, chemotherapy poisoning, poor vision, peripheral artery disease, smoker's leg, diabetic feet, periodontal disease, varicose or spider veins, blood clots, COVID-19, long COVID, liver disease, symptoms caused by chemotherapy or radiation therapy, hair loss, cancer, inflammation-caused or inflammation-related diseases, autoimmune diseases, infections, and headaches including migraines.

Methods of preventing or treating diseases using a preparation as recited above are disclosed. Additional methods include applying an oil to the nasal membrane or atomizing the oil and inhaling the mist.

### DETAILED DESCRIPTION

The compound YEL002 has a myriad of medicinal and other therapeutic properties, some of which were previously described in U.S. Pat. No. 9,045,474 titled “Compounds and compositions for mitigating tissue damage and lethality.” The patent discloses embodiments for various formulations containing YEL002. Embodiments as to use, dosage, and effects are described herein but all uses are included and not limited by the descriptions.

The molecular structure of the YEL002 compound is described below:

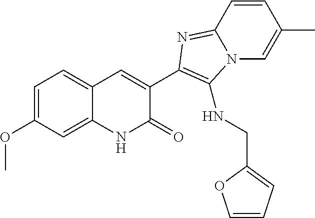 [SMILES: COc1ccc2cc(-c3nc4ccc(C)cn4c3NCc3ccco3)c(=O)[nH]c2c1]

The dose to use is about 1 gram per day for a 70 kg person which is the same as was used in previous experiments with mice. A treatment of 20 mg/kg, a quarter teaspoon or about 0.5 g dissolved in oil is effective.

The compound takes the appearance of an off-white powder and is fat soluble. One takes a quarter teaspoon or about 0.5 g dissolved in a couple of teaspoons of oil. In an exemplary embodiment, it is dissolved in grape seed oil and taken twice a day, once in the morning and once in the evening. It is easy to use and has almost no taste.

The compound can also be delivered into the bloodstream through the nose as a nose spray. The compound is dissolved in oil and sprayed into or applied inside the nose. Furthermore, YEL002 can be applied just by itself or during inhalation of a mist produced by atomizing the oil and inhaled. All other delivery methods, such as the powder by itself, in pills, preferably sublingual pills or as injections, are also contemplated.

YEL002 has a huge number of uses and holds promise as a most useful future pharmaceutical. In a high-throughput cellular screen at UCLA for a radiation mitigator funded by the National Institute of Health with $31 million during 10 years, 16,000 compounds from different chemical libraries were screened.

There were 10 primary hits and 6 confirmed hits and 2 of them were used in mice. Mice were irradiated with an LD100 after which 100% of the mice died after 10 to 15 days. When the researchers injected YEL002 IP into the irradiated mice 24 hours after radiation and another four doses in 24 hours intervals 100% of the mice, so all mice which would otherwise have died survived which was a highly significant difference. Many lived a normal life span of more than 2 years which is interesting since other labs which isolated other compounds their mice died of late radiation effects much earlier.

It is speculated that this is because the primary screen applied was against radiation-induced toxicity as well as genotoxicity which causes cancer. Researchers involved in other labs were screening only against radiation-induced toxicity and thereby cells with genotoxicity survive and cause even more cancer and late effects of which surviving mice died. In our experiments, however, a screen against radiation induced toxicity as well as genetic instability made sure that the surviving cells also had no genetic instability which otherwise would have led to an increase in cancer and other diseases. In the experiment, ATM-deficient mice injected every week with YEL002 during most of their lifespans lived 33% longer than without injections.

In another experiment at the National Institute of Health, in other mice after radiation exposure which caused 90% of them to develop lymphoma, there was a reduction of the lymphoma incidence to 40% and the spontaneous frequency was reduced from 10-20% down to 0%. The compound was shown to work in yeast, human tissue culture cells, zebrafish and mice. Extrapolating these results to humans, it is speculated that humans could live 20 years longer and with a reduction of cancer and probably many degenerative diseases as well as cancer. This was observed after treating mice right after birth, if humans are treated later in life it is possible that only 10 or 5 years are added and probably more for DNA repair deficient diseases like ataxia telangiectasia (ATM) which is still very substantial.

The compound made cells more resistant to exposure to a variety of agents, such as UV, chemical carcinogens, chemical warfare agents, radioactive iodine and cigarette smoke.

In case of radiation accidents or nuclear reactor accidents such as Fukushima or Chernobyl, or exposure to a nuclear or a dirty bomb, for the army against chemical warfare agents like in the Iran-Iraq war, for stewardesses, pilots and astronauts which have a higher occupational risk of cancer and frequent fliers or any other flier against space radiation effects, against radon in the basement induced lung cancer, against exposure to all kinds of carcinogens induced cancer and other diseases, against smoking and most likely also air pollution induced cancer or other diseases (truck drivers, police officers, inner city dwellers, etc.). And since cells became resistant to a variety of agents, it is speculated that there is a limitless number of experiments to be done to identify which additional agents YEL002 makes cells or animals resistant to.

It is also observed that three different cancer cell lines were not protected from radiation killing by YEL002, which indicates a use in radiation oncology or chemotherapy where the tumor can still be killed by the radiation, or chemotherapy but YEL002 protects the rest of the body from radiation or chemotherapy damage, and inhibits secondary carcinogenesis. There were no side effects besides longer life and reduction of cancer incidence and protection from exposure to radiation and many carcinogens and many other beneficial effects.

In terms of mechanism of action in a proteomics study, the chemical was capable of inducing 244 different proteins in human keratinocytes, which comprised anti-inflammatory proteins, a number of DNA repair proteins, and even some neuroprotective proteins. It is known that turtles at the Galapagos Islands live 150 or more years without cancer and have multiple copies of DNA repair genes and anti-inflammatory genes. The compound boosts the expression of these genes in mammals. Since it induces DNA repair and is anti-inflammatory, it may be possible to prevent and cure many diseases and extend the life.

51% of the population die from inflammation-related diseases and 6% of the population have autoimmune diseases, and 9% have infectious diseases. It is estimated that 5.5 billion of the world population may potentially benefit from YEL002 treatment.

Case studies show that COVID disease was treated and the Covid virus was cleared faster than without YEL002 and it supports the immune system to fight the virus and may also reduce the risk of infection. It may cure inflammation as well as autoimmune diseases and maybe even infections. There are 16 million new cases of COVID worldwide each week and 600 million cases of late COVID in the world which are due to continued inflammation and autoimmune diseases which also should be treatable.

A case study where it was used for eight weeks reduced symptoms of coughing and diarrhea, reducing the needs for drugs to treat those symptoms. Reportedly the compound was able to straighten and strengthen teeth such that the subject does not catch much food in their teeth any longer. It has also beneficial effects on the skin and one needs less of a cosmetic cream containing the same compound.

In a user study, the patient's periodontics or receding gums were healed and no longer needed to floss teeth as much, since the teeth are now perfectly aligned and close together without a space in between.

The patient also observed no longer needing eyeglasses after 55 years of wearing them, and after 4 weeks with the drug, the subject can see much better than anybody his age. According to the patient, the compound fixed 2 diopters and 1 astigmatism of eye sight deficiency in only 4 weeks. An eye exam was conducted in an ophthalmology office. In one subject 2 diopters and one astigmatism completely vanished and another subject had 6 diopters and does not require eyeglasses any longer. In every subject using the chemical the eye background is completely green, because YEL002 benefits the retina and optic nerve.

According to patient testimonial, his arms and legs are tighter and nurses have problems finding a vein for the first time in his life, since the veins moved further inside the extremities and are not on the surface any longer, thus it fixes varicose and spider veins. His blood is thinner, since proinflammatory factors make platelets sticky and cause blood clots. Thus, it prevents or treats blood clots.

According to another patient testimonial, one subject had twice elevated levels of liver damage ever since she had a gallbladder operation 25 years ago. After only two days of YEL002 treatment the values were normal and below the cutoff for the first time in 25 years as was her cholesterol.

According to another patient testimonial, one subject on chemotherapy was repeatedly vomiting many times a day, had no appetite, continually lost weight, did not eat, and did not want to live any longer. One week on YEL002 vomiting completely stopped, he had lots of appetite and is eating, gained 2 kg, and drove 320 km with his motorbike. He is extremely happy and all his doctors are happy too and told him that he needs to continue his experimental therapy.

According to another patient testimonial, one subject post-chemotherapy had coughing and wheezing symptoms and went downhill very fast and her daughter was worried that she was dying. After only one hour after YEL002 treatment this was completely alleviated and she slept well.

According to another patient testimonial, one subject was a heavy smoker and she had all kinds of inflammation problems including smoker's leg which were oozing lymph liquid and could not heal for many years. One week after YEL002 treatment her condition was completely healed and for the first time in years her brush did not have any hair, meaning her hair loss completely stopped.

According to another patient testimonial, one subject dissolved YEL002 in oil and used that oil to spray her nose 2-4 times a day. This felt great and completely prevented all allergic attacks such as sneezing and runny nose.

Since diabetics have the same problem with their feet this could be fixed too, so that nobody must amputate feet or legs instead of using YEL002.

Based on the testimonials, it is apparent that the compound may be efficacious for varicose veins, spider veins, anti-inflammatory effects to prevent or treat blood clots, liver problems due to autoimmune diseases or excessive drinking.

In addition, patients are using the compound to try to treat Alzheimer's, multiple sclerosis, alopecia, diabetes, to prevent prostate cancer, to increase testosterone level, etc. People report feeling more energetic, less tired, and less combative and become nicer. The actual mechanism is not well defined, but may either be because the entire body is working better without pains, or because proteomics shows that YEL002 induces 9 neuroprotective functions amongst DNA repair and anti-inflammatory functions.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

1. A composition comprising a preparation of YEL002 as an active ingredient for the prevention or treatment of one or more diseases in humans.

2. The preparation of claim 1, wherein YEL002 is used by itself.

3. The preparation of claim 1, wherein the preparation is a sublingual pill.

4. The preparation of claim 1, wherein the preparation is an injection.

5. The preparation of claim 1, wherein the preparation is dissolved in a liquid.

6. The preparation of claim 1, wherein the preparation is dissolved in an oil.

7. The preparation of claim 6, wherein the oil is grapeseed oil.

8. The preparation of claim 1, wherein the disease is one or more of:
poisoning;
radiation poisoning;
chemotherapy poisoning;
poor vision;
peripheral artery disease;
smoker's leg;
diabetic feet;
periodontal disease;
varicose or spider veins;
blood clots;
COVID-19;
long COVID;
liver disease;
symptoms caused by chemotherapy or radiation therapy;
hair loss;
cancer;
inflammation-caused or inflammation-related diseases;
autoimmune diseases;
infections; and
headaches.

9. A method of preventing or treating one or more diseases in humans, said method comprising administering a composition comprising a preparation of YEL002 to a patient at a predetermined dosage.

10. The method of claim 9, wherein the dosage is 0.01 to 5 grams per 70 kg body weight per day.

11. The method of claim 10, wherein the dosage is 1.0 grams per 70 kg body weight per day.

12. The method of claim 9, wherein the preparation is a sublingual pill.

13. The method of claim 9, wherein the preparation is an injection.

14. The method of claim 9, wherein the preparation is YEL002 dissolved in liquid.

15. The method of claim 9, wherein the preparation is YEL002 dissolved in an oil.

16. The method of claim 15, wherein the oil is grapeseed oil.

17. The method of claim 9, wherein administering the preparation further comprises applying the oil containing YEL002 to the nasal membrane.

18. The method of claim 15, wherein administering the preparation further comprises atomizing the oil containing YEL002 and inhaling the mist.

19. The method of claim 9, wherein the disease is one or more of:
poisoning;
radiation poisoning;
chemotherapy poisoning;
poor vision;
peripheral artery disease;
periodontal disease;
smoker's leg;
diabetic feet;
varicose or spider veins;
blood clots;
COVID-19;
long COVID;
liver disease;
symptoms caused by chemotherapy or radiation therapy;
hair loss;
cancer;
inflammation-caused or inflammation-related diseases;
autoimmune diseases;
infections; and
headaches.
